(19) United States
(12) Patent Application Publication
(10) Pub. No.: US 2026/0106029 A1
(43) Pub. Date: Apr. 16, 2026

(54) 2-D POSE ESTIMATION-BASED AUTOMATED TRUNK/THORACIC RANGE OF MOTION MEASUREMENT TOOL

(71) Applicant: My Medical HUB Corporation, Safety Harbor, FL (US)

(72) Inventors: Walter M Groteke, Safety Harbor, FL (US); Barry Fitch, North Augusta, SC (US); Md. Al Mehedi Hasan, Rajshahi (BD); Rifaet Ullah, Rangpur (BD)

(21) Appl. No.: 19/413,858

(22) Filed: Dec. 9, 2025

Related U.S. Application Data
(63) Continuation-in-part of application No. 19/217,777, filed on May 23, 2025, now Pat. No. 12,505,928.
Application No. 19/217,777 is a division of application No. 18/794,726, filed on Aug. 5, 2024, now Pat. No. 12,315,637.
Application No. 18/794,726 is a continuation-in-part of application No. 17/806,874, filed on Jun. 14, 2022, now Pat. No. 12,057,212.
Application No. 17/806,874 is a continuation-in-part of application No. 16/913,236, filed on Jun. 26, 2020, now abandoned.
(60) Provisional application No. 62/866,786, filed on Jun. 26, 2019.
(60) Provisional application No. 63/210,623, filed on Jun. 15, 2021.
(60) Provisional application No. 63/582,544, filed on Sep. 14, 2023.

(51) Int. Cl. G16H 50/20

(52) U.S. Cl. CPC G16H 50/20

(57) ABSTRACT

A method of remotely measuring a patient's range of motion using a device having a camera, the device being in data communication with a computer system having a database and an AI bot in communication with the database, the method having: instructing the patient through a stretching procedure; recording a video of the patient as they proceed through the stretching procedure, the video having frames of the patient in a rest pose and a fully stretched pose; capturing the frames of the patient in the rest pose and the fully stretched pose; identifying key-points within the frames; tracing range of movement for the key-points in the frames; calculating a range of motion angle for the stretching procedure by comparing key-point positions between the frames; recording the range of motion angle in the database; and reporting the range of motion angle to the patient or medical personnel.

### CROSS-REFERENCE TO RELATED APPLICATIONS

This application is a continuation-in-part of U.S. Non-Provisional application Ser. No. 19/217,777, filed Mar. 23, 2025, which is a divisional and claims the benefit of U.S. Non-Provisional application Ser. No. 18/794,726, filed Aug. 5, 2024, which claims the benefit of U.S. Provisional application No. 63/582,544, filed Sep. 14, 2023, and is a continuation-in-part and claims the benefit of U.S. Non-Provisional application Ser. No. 17/806,874 filed Jun. 14, 2022, which claims the benefit of U.S. Non-Provisional application Ser. No. 16/913,236, filed Jun. 26, 2020, which claims the benefit of U.S. Provisional Application No. 62/866,786, filed Jun. 26, 2019, and claims the benefit of U.S. Provisional Application No. 63/210,623, filed Jun. 15, 2021, all of which are hereby incorporated by reference, to the extent that they are not conflicting with the present application.

### BACKGROUND OF INVENTION

### 1. Field of the Invention

The invention relates generally to systems and methods for analyzing patients and specifically to systems and methods for pose estimation for automatically measuring a patient's range of motion.

### 2. Description of the Related Art

In order to assess a patient's overall health, clinicians will often measure different aspects of the patient's physical capabilities, including the patient's range of motion. A commonly utilized tool for the measurement of a patient's range of motion is a manual goniometer. Despite their common utilization, mechanical tools such as the manual goniometer may have several notable limitations that may limit their usefulness. The currently utilized methodology of measuring a patient's range of motion (“ROM”) using a manual goniometer may be inaccurate, difficult to perform, and prone to wide ranges of variation. Furthermore, the manual goniometer, and other similar manual measurement devices, require the clinicians to be on site with the patient, thus removing the potential for said measurements to be acquired remotely without someone to assist the patient. This lack of access to remote methods for measuring a patient's range of motion may limit the quality of care provided to a patient utilizing telemedicine or other types of remote care.

Therefore, there is a need to solve the problems described above by proving a device and method for remotely taking range of motion measurements for a patient through the utilization of 2-D pose estimation techniques.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF INVENTION SUMMARY

This Summary is provided to introduce a selection of concepts in a simplified form that are further described below in the Detailed Description.

In an aspect, a method of remotely measuring a patient's thoracolumbar rotation range of motion using a device having a camera is provided, the device being in data communication with a computer system, the computer system having a database and an AI bot in data communication with the database, the method comprising: instructing the patient through a thoracolumbar rotation stretching procedure by providing verbal instructions from the AI bot to the patient; recording a video of the patient as they proceed through the stretching procedure, the video having at least a frame of the patient in a rest pose, a frame of the patient in a fully stretched pose and a frame of the patient in an intermediate pose between the rest pose and the fully stretched pose; capturing the frames of the patient in the rest pose, the fully stretched pose and the intermediate pose from the video; identifying key-points within the frames, the key-points comprising at least a corresponding acromion process of the patient; tracing range of movement for the key-points in the frames; calculating a range of motion angle for the stretching procedure by comparing key-point positions between the frames; recording the range of motion angle in the database; and reporting the range of motion angle to the patient or medical personnel. Thus, an advantage is that a patient's range of motion may be measured accurately using a camera on the device without the use of auxiliary sensors, tools, or other devices. Another advantage is that a patient's range of motion may be measured remotely without the need for on-site personnel, thus allowing a user to take certain kinds of range of motion assessments from home. Another advantage is that the collected range of motion angles(s) may be provided to the patient/provider/medical personnel as part of a comprehensive report, wherein additional medical information may be provided in relation to the measured range of motion angle(s). Another advantage is that the disclosed method may be further comprised of asking the patient other questions relevant to their health, in order to provide a more thorough assessment of the patient's overall health. Another advantage is that an AI bot may be utilized to instruct patients through the stretching procedure, thus helping to automate the process of measuring the patient's range of motion.

In another aspect, a method of remotely measuring a patient's range of motion using a device having a camera is provided, the device being in data communication with a computer system, the computer system having a database and an AI bot in data communication with the database, the method comprising: instructing the patient through a stretching procedure; recording a video of the patient as they proceed through the stretching procedure, the video having at least a frame of the patient in a rest pose and a frame of the patient in a fully stretched pose; capturing the frames of the patient in the rest pose and the fully stretched pose from the video; identifying key-points within the frames; tracing range of movement for the key-points in the frames; calculating a range of motion angle for the stretching procedure by comparing key-point positions between the frames; recording the range of motion angle in the database; and reporting the range of motion angle to the patient or medical personnel. Again, an advantage is that a patient's range of motion may be measured accurately using a camera on the device without the use of auxiliary sensors, tools, or other devices. Another advantage is that a patient's range of motion may be measured remotely without the need for on-site personnel, thus allowing a user to take certain kinds of range of motion assessments from home. Another advantage is that the collected range of motion angles(s) may be provided to the patient/provider/medical personnel as part of a comprehensive report, wherein additional medical information may be provided in relation to the measured range of motion angle(s). Another advantage is that the disclosed method may be further comprised of asking the patient other questions relevant to their health, in order to provide a more thorough assessment of the patient's overall health. Another advantage is that an AI bot may be utilized to instruct patients through the stretching procedure, thus helping to automate the process of measuring the patient's range of motion.

In another aspect, a method of remotely measuring a patient's range of motion using a device having a camera is provided, the device being in data communication with a computer system, the computer system having a database, the method comprising: instructing the patient through a stretching procedure; recording a video of the patient as they proceed through the stretching procedure, the video having at least a frame of the patient in a rest pose and a frame of the patient in a fully stretched pose; capturing the frames of the patient in the rest pose and the fully stretched pose from the video; identifying key-points within the frames; tracing range of movement for the key-points in the frames; calculating a range of motion angle for the stretching procedure by comparing key-point positions between the frames; recording the range of motion angle in the database; and reporting the range of motion angle to the patient or medical personnel. Again, an advantage is that a patient's range of motion may be measured accurately using a camera on the device without the use of auxiliary sensors, tools, or other devices. Another advantage is that a patient's range of motion may be measured remotely without the need for on-site personnel, thus allowing a user to take certain kinds of range of motion assessments from home. Another advantage is that the collected range of motion angles(s) may be provided to the patient/provider/medical personnel as part of a comprehensive report, wherein additional medical information may be provided in relation to the measured range of motion angle(s). Another advantage is that the disclosed method may be further comprised of asking the patient other questions relevant to their health, in order to provide a more thorough assessment of the patient's overall health. Another advantage is that an AI bot may be utilized to instruct patients through the stretching procedure, thus helping to automate the process of measuring the patient's range of motion.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1A-1C illustrates the procedure for a patient performing a right thoracolumbar rotation range of motion measurement test, according to an aspect.

FIG. 1D illustrates an image captured by a camera being utilized to record the right thoracolumbar rotation range of motion measurement test for a patient, according to an aspect.

FIG. 2A-2C illustrates the procedure for a patient performing a left thoracolumbar rotation range of motion measurement test, according to an aspect.

FIG. 2D illustrates an image captured by a camera being utilized to record the left thoracolumbar rotation range of motion measurement test for a patient, according to an aspect.

FIG. 3 illustrates the design diagram for the MMH (MyMedicalHUB) system, according to an aspect.

### DETAILED DESCRIPTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention.

As used herein and throughout this disclosure, the term “mobile device” refers to any electronic device capable of communicating across a mobile network. A mobile device may have a processor, a memory, a transceiver, an input, and an output. Examples of such devices include cellular telephones, personal digital assistants (PDAs), portable computers, etc. The memory stores applications, software, or logic. Examples of processors are computer processors (processing units), microprocessors, digital signal processors, controllers and microcontrollers, etc. Examples of device memories that may comprise logic include RAM (random access memory), flash memories, ROMS (read-only memories), EPROMS (erasable programmable read-only memories), and EEPROMS (electrically erasable programmable read-only memories). A transceiver includes but is not limited to cellular, GPRS, Bluetooth, and Wi-Fi transceivers.

“Logic” as used herein and throughout this disclosure, refers to any information having the form of instruction signals and/or data that may be applied to direct the operation of a processor. Logic may be formed from signals stored in a device memory. Software is one example of such logic. Logic may also be comprised by digital and/or analog hardware circuits, for example, hardware circuits comprising logical AND, OR, XOR, NAND, NOR, and other logical operations. Logic may be formed from combinations of software and hardware. On a network, logic may be programmed on a server, or a complex of servers. A particular logic unit is not limited to a single logical location on the network.

Mobile devices communicate with each other and with other elements via a network, for instance, a cellular network. A “network” can include broadband wide-area networks, local-area networks, and personal area networks. Communication across a network can be packet-based or use radio and frequency/amplitude modulations using appropriate analog-digital-analog converters and other elements. Examples of radio networks include GSM, CDMA, Wi-Fi and BLUETOOTH® networks, with communication being enabled by transceivers. A network typically includes a plurality of elements such as servers that host logic for performing tasks on the network. Servers may be placed at several logical points on the network. Servers may further be in communication with databases and can enable communication devices to access the contents of a database. For instance, an authentication server hosts or is in communication with a database having authentication information for users of a mobile network. A “user account” may include several attributes for a particular user, including a unique identifier of the mobile device(s) owned by the user, relationships with other users, call data records, bank account information, etc. A billing server may host a user account for the user to which value is added or removed based on the user's usage of services. One of these services includes mobile payment. In exemplary mobile payment systems, a user account hosted at a billing server is debited or credited based upon transactions performed by a user using their mobile device as a payment method.

For the following description, it can be assumed that most correspondingly labeled elements across the figures (e.g., 100 and 200, etc.) possess the same characteristics and are subject to the same structure and function. If there is a difference between correspondingly labeled elements that is not pointed out, and this difference results in a non-corresponding structure or function of an element for a particular embodiment, example or aspect, then the conflicting description given for that particular embodiment, example or aspect shall govern.

It should be understood that, for clarity of the drawings and of the specification, some or all details about some structural components or steps that are known in the art are not shown or described if they are not necessary for the invention to be understood by one of ordinary skills in the art.

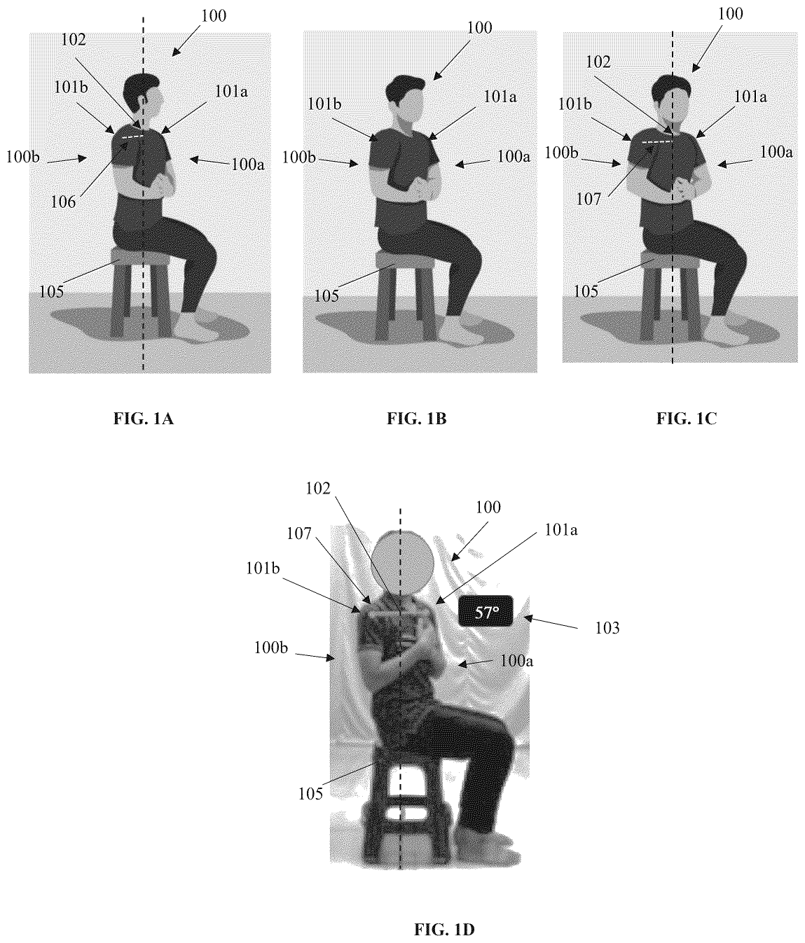

FIG. 1A-1C illustrate the procedure for a patient 100 performing a right thoracolumbar rotation range of motion measurement test, according to an aspect. FIG. 1D illustrates an image captured by a camera being utilized to record the right thoracolumbar rotation range of motion measurement test for a patient 100, according to an aspect. In order for a patient 100 to have their range of motion measured for a particular stretching procedure/range of motion test (“ROM” test”), the patient 100 may utilize a specific tool within a computer system called “MyMedicalHUB”. As disclosed hereinbelow, MyMedicalHUB has developed a standardized, automated tool that uses 2-D (“two-dimensional”) pose estimation to gather and generate ROM data in an unbiased manner. As a result of these herein disclosed stretching procedures being executed by the patient without the physical assistance/provocation of a clinician/physician, said ROM measurements collected may be referred to as “active” ROM measurements, or “AROM” measurements. It should be understood that while embodiments detailed hereinbelow may be configured to capture AROM measurements, the techniques and methods disclosed herein may also be configured to suitably capture other ROM measurement, such as “passive” range of motion measurements, in which a patient may be physically assisted by a clinician. It should also be understood that a patient 100 may perform a particular stretching procedure during a corresponding ROM test in order to assess the patient's flexibility for a particular part of their body, as will be described in greater detail hereinbelow.

MyMedicalHUB (MMH) is a cloud-based technology that automates and captures data that has been traditionally generated manually by clinicians. In an embodiment, patients are invited by a clinician to log-in to MyMedicalHUB via a secure link, register, and complete a comprehensive online physical assessment. The assessment includes subjective intake questions consistent with in-office evaluation and management examinations, such as Activities of Daily Living (ADLs), personal and family medical history, chief complaint(s), and, in some cases, subjective survey tools, such as an Oswestry Disability Index or STEADI Fall Risk Assessment Questionnaires. Once the subjective intake questions are complete, the patient 100 may be led through a series of provocative movements (e.g., a stretching procedure) to measure and record their range of motion using a computer, tablet, cell phone, smartphone, smart device, mobile device, or other device having a two-dimensional video camera (e.g., a camera configured to capture standard, two dimensional video footage), as opposed to a traditional goniometer and other sensor or device related signaling technology. In an embodiment, the MMH system may be configured to guide the patient through the correct stretching procedure through the utilization of an “Efficient Musculoskeletal Management Assistant” (“EMMA”) system. This EMMA system may utilize bot synthesized voice technology to verbally instruct patients through the corresponding stretching procedures to acquire a desired ROM measurement, wherein said synthesized voice may be played through the same device having the camera, or an auxiliary audio device.

It should be understood that the device utilized to capture a video of the patient performing the stretching procedure may comprise a processor, a user interface and a camera, to facilitate the suitable capture of all required information. It should be understood that the term “frame” and “picture” may be utilized interchangeably herein, wherein the frames/pictures correspond to the individual images that make up a recorded video. In an embodiment, the movements of the stretching procedure may be tailored to measure a patient's trunk rotation ROM. In an embodiment, the ROM measurements that are measured from the patient may be compared to normative ranges, as well as used to track objective data on a longitudinal basis. Following each stretching procedure, MMH may also ask the patient specific questions that encompass symptomatic criteria outlined in published, peer reviewed clinical studies and evidenced-based medicine protocols.

The MMH system may utilize two novel processes for measuring a patient's range of motion for a selected stretching procedure, such as trunk rotation. First, a unique process was developed for obtaining the measurement; and secondly, the MMH has been developed to utilize a special methodology for interpreting the corresponding technology's AI observations. For right thoracolumbar rotation ROM measurements, a patient may sit in a chair 105 with their right side facing the camera, cross their arms, interlock their fingers adjacent to their naval, and rotate their shoulders in unison in a clockwise direction while keeping their hips stationary, i.e., forward facing, as seen in FIG. 1A-1C. The MMH system's video AI technology may be configured to capture the movement at maximum range/value on a recorded video, and superimpose virtual markers on specific key-points of the patient 100, trace the range of movement for said key-points (e.g., trace the movement of the key-points between relevant video frames), calculate the angle at the maximum point of the movement, measure it in degrees, and feed the information into a comprehensive report for the patient and/or provider. This comprehensive report containing the patient's ROM angle(s), as well as the recorded videos and corresponding frames/pictures of the video from which they were calculated, may be stored on a corresponding database within the MMH system, for later use or reference.

In order to provide the MMH system with the required images to accurately measure a patient's range of motion for a particular stretching procedure, it is important for both the patient performing stretching procedure and a clinician observing the range of motion test, if applicable, to follow the prescribed procedure for measuring said patient's range of motion. In an embodiment, the ROM test being performed may be configured to measure the right thoracolumbar rotation range of motion for a patient 100. In order to perform this range of motion test, first a patient 100 may sit on a chair height stool 105 (approximately 18″ high, in an embodiment) with their right side 100b squarely facing a camera. Next, the patient 100 may interlock their hands just above navel height and sit upright to the best of their ability, resetting their shoulders backward, and assuming a rest pose, as seen in FIG. 1A. After assuming the rest pose of FIG. 1A as described above, the patient may then turn their shoulders and trunk as far to the right (clockwise) as possible without moving their shoulders or hips, and then hold this pose until corresponding measurements are taken (e.g., the video is recorded), as seen in FIG. 1C. A frame for an intermediate pose, as shown in FIG. 1B, may also be captured from the recorded video, which may be helpful in tracing the movement of key-points on the patient from the rest pose of FIG. 1A to the fully stretched pose of FIG. 1C.

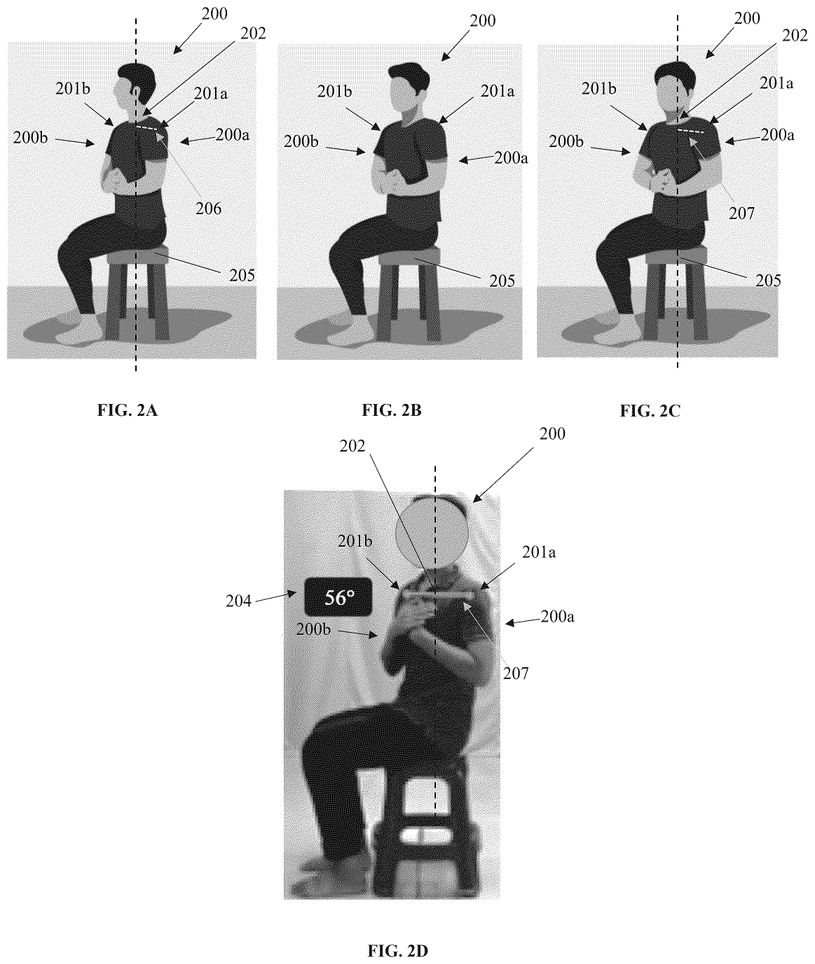

The capture of intermediate poses from the recorded video, such as the intermediate pose for the right thoracolumbar rotation ROM stretching procedure, as seen in FIG. 1B, may be particularly relevant for detecting, tracking and compensating for any “drift” that may occur during the stretching procedure. Drift is a phenomenon that may occur when the patient 100 adjusts themselves to compensate for a lack of movement and/or rotation for a particular part of their body. This drift, if left unaccounted for, may negatively influence the accuracy of ROM measurements. As such, the MMH system may be configured to detect, track and compensate for drift through utilization of captured intermediate poses, such as the intermediate poses of FIG. 1B and FIG. 2B. For example, movement of a patient's hips observed within an intermediate pose of the disclosed right thoracolumbar rotation ROM stretching procedure may be indicative of drift occurring during said stretching procedure, which the MMH system is configured to compensate for in order to facilitate accurate ROM measurements. As such, the MMH system may be configured to analyze a captured intermediate frame of the video captured during an intermediate pose to determine if drift has occurred, and compensate for this drift through suitable calculations to ensure the accuracy of the ROM measurement is not influenced by the drift that occurred.

While the patient is performing the above described process, a provider/clinician may also follow a corresponding procedure in order to aid the patient 100 in performing the stretching procedure, such as through verbal instruction. It should be understood that the instructions provided to the patient 100 to guide them through the corresponding stretching procedure may be delivered to them by a clinician observing the patient (remotely or otherwise) and/or by the bot/EMMA system disclosed hereinabove. The instructions may be delivered to the patient 100 in an outpatient clinical setting or outside an office setting, through corresponding telehealth-enabled technologies. In an embodiment, the provider/clinician may make sure that the patient is lined up properly with the camera while they are performing the corresponding range of motion procedure to ensure accurate frame capture. The provider/clinician may also make sure that the patient sits upright and minimizes their hip movement while turning to the best of their ability to ensure accurate results may be obtained. In an embodiment in which the stretching procedure is not performed satisfactorily by the patient, the provider/clinician may instruct the patient to repeat the stretching procedure while providing clarifying instruction to ensure the stretching procedure is performed properly. Additionally, the provider/clinician may also indicate corrective actions required during the stretching procedure, rather than indicating that the whole stretching procedure needs to be redone. This instructing of the patient to repeat stretching procedures may be similarly done by EMMA/AI bot, which may also observe the patient through the camera of a corresponding device to ensure the stretching procedure is performed properly.

During the stretching procedure described hereinabove, the MMH system may be configured to automatically operate the camera to capture video of the patient in the rest pose of FIG. 1A and the fully stretched pose of FIG. 1C, as well as the patient in the intermediate pose of FIG. 1B, as applicable. As shown in FIG. 1D, this ROM measurement for the right thoracolumbar rotation may be measured and displayed within the MMH system. In the embodiment of FIG. 1D, the patient's ROM angle 103 for their right thoracolumbar rotation may be about 56°. The MMH system may be configured to measure this ROM angle and provide it on a comprehensive report for later reference by the patient 100 and/or a provider/clinician. As is understood, the herein disclosed process for measuring a patient ROM's may utilize two-dimensional pose estimation to gather and generate relevant results.

In describing the disclosed range of motion measurement process for a particular stretching procedure, it is useful to establish a “Goniometric Protocol” utilized to analyze the captured images. For a right thoracolumbar rotation range of motion test, an “axis” (“axis of rotation”) 102 may be identified as the center point of a straight line between acromion processes 101a, 101b of the shoulders of the patient 100 as measured from above the head. Furthermore, a “fixed arm” 106 may remain parallel to the original position of the right acromion process 101b in the rest pose of FIG. 1A, whereas a “moving arm” 107 may move to point to the new position of the right acromion process 101b in the fully stretched pose of FIG. 1C. With both the original position and new position of the patient's right acromion process 101b known from measurements made to corresponding pictures/frames of the recorded video, the patient's range of motion for right thoracolumbar rotation may be determined, as indicated by ROM measurement (“ROM angle”) 103 in FIG. 1D. The disclosed process measures a series of ranges, key-points, and lengths within corresponding frames of the captured video, translates them into digitized data points, and measures the movement of these data points using a corresponding methodology, based on the ROM measurement being collected.

In order to enable the calculation of the hereinabove described ROM measurement 103 for the corresponding procedure, the MMH system may follow a corresponding process. When a new patient first accesses MMH, they may be directed through a registration via a link, if they are outside of a clinic, or during an in-office encounter. Once complete, the MMH system establishes a secure, individual portal for every patient assessed. Once established, patients can reenter their portal via a web link or saved URL, access information pertaining to their assessments, including exercises, and conduct new assessments, if ordered by the provider. The patient may be allowed to position a device having a camera in the correct position to allow for suitable video capture, such that ROM measurements may be obtained. Then, the patient may be instructed on how to perform the necessary stretching procedure to calculate the desired ROM angle. In an embodiment, this instructing of the patient may be done as an automated aspect of the MMH system, such as through bot delivered instructions, wherein a clinician is absent, and the stretching procedure is led by the EMMA system/bot (e.g., a self-led bot assessment.) In an alternative embodiment, the clinician/provider may verbally guide the patient 100 through the steps of the stretching procedure. While the stretching procedure is being performed, the MMH system may be configured to automatically record a video of the patient 100 using the camera, and identify and extract frames corresponding to the pertinent poses for ROM measurements. These frames may include at least a frame of the patient in the rest pose, as seen in FIG. 1A, and a frame of the patient at their maximum range of motion/maximum extension, called a fully stretched pose, as seen in FIG. 1C.

With the necessary frames of the patient 100 performing the stretching procedure obtained from the recorded video, the ROM measurement procedure may continue with the identification of the analytic elements, such as the axis 102, fixed arm 106 and moving arm 107, within the recorded frames. As described hereinabove, the identification of the axis, fixed arm and moving arm may be done through the tracking of relevant key-points on the patient within the corresponding frames, such as their right acromion process 101b and their left acromion process 101a for left or right thoracolumbar range of motion measurements. In an embodiment, the axis 102 itself may be described as a key-point in the angle difference calculation, since it is utilized in the identification of the fixed arm 106 and moving arm 107. With the fixed arm 106, moving arm 107 and axis 102 identified, the ROM angle 103 may be calculated by measuring the angle difference between the moving arm 106 and the fixed arm 107. In an embodiment, the fixed arm 106 may be formed between the axis 102 and the right acromion process 101b while in the rest position of FIG. 1A, whereas the moving arm 107 may be formed between the axis 102 and the right acromion process 101b while in the fully stretched position of FIG. 1C position. With the right thoracolumbar range of motion angle 103 calculated, the right thoracolumbar range of motion angle 103 may be reported to the patient/clinician, as well as recorded on a comprehensive health report for the patient/clinician. It should be understood that the MMH system may be configured to be utilized to measure any necessary ROM angles by recording/observing the patient 100 perform the corresponding stretching procedure while also identifying the positions of relevant /y-points/ key analytic elements, accordingly. This process of identifying a patient's range of motion for a particular stretching procedure may also be done for other stretching procedures for other ROM tests, such as left thoracolumbar rotation ROM test, as disclosed hereinbelow.

FIG. 2A-2C illustrates the procedure for a patient 200 performing a left thoracolumbar rotation range of motion measurement test, according to an aspect. FIG. 2D illustrates an image captured by a camera being utilized to record the left thoracolumbar rotation range of motion measurement test for a patient 200, according to an aspect. As disclosed hereinabove, the patient 200 may access the MMH system in order to utilize the herein disclosed left thoracolumbar rotation range of motion measurement test.

In an embodiment, the process utilized to capture the left thoracolumbar rotation ROM may mirror the process utilized to capture the right thoracolumbar rotation ROM, as described in FIG. 1A-1D above, with a few exceptions. In order to perform this range of motion test, first a patient 200 may sit on a chair height stool 205 (approximately 18″ high, in an embodiment) with their left side 200a squarely facing a camera. Next, the patient 200 may interlock their hands just above navel height and sit upright to the best of their ability, resetting their shoulders backward, thus assuming a rest pose for the left thoracolumbar rotation ROM test, as seen in FIG. 2A. After assuming this rest pose, the patient may then turn their shoulders and trunk as far to the left (counterclockwise) as possible without moving their shoulders or hips, and then hold this pose until corresponding measurements are taken (e.g., the video is recorded), as seen in FIG. 2C. An intermediate pose for the left thoracolumbar rotation range of motion measurement test, as shown in FIG. 2B, may also be captured if helpful or necessary. As disclosed hereinabove, the intermediate pose from a stretching procedure may be utilized to help detect, track, and compensate for drift during a ROM test. As with the previous ROM measurement embodiment, frames will be identified from the captured video for at least the extreme values, such as the rest pose of FIG. 2A and the fully stretched pose of FIG. 2C, to determine the patient's ROM for the left thoracolumbar rotation stretching procedure. Aside from the changes disclosed hereinabove, and the measurements being taken for the patient's left side 200a instead of their right side 200b, the process for measuring a patient's left thoracolumbar rotation ROM may be largely the same as the process for measuring a patient's right thoracolumbar rotation ROM.

Similarly to the right thoracolumbar rotation range of motion measurement test, the left thoracolumbar rotation range of motion measurement test may establish a goniometric protocol in order to suitably establish the information to be collected to assess the patient's range of motion for said procedure. Again, an “axis” 202 may be identified as the center point of a straight line between acromion processes 201a, 201b of the patient's shoulders as measured from above the head. Furthermore, the “fixed arm” 206 may remain parallel to the original position of the left acromion process 201a in the rest pose of FIG. 2A, whereas a “moving arm” 207 may move to point to the new left acromion process 201a position in the fully stretched pose of FIG. 2C. With both the original position and new position of the patient's left acromion process 201a known from measurement of the recorded frames, the range of motion for the patient's left thoracolumbar rotation may be determined, as indicated by angle measurement 204 in FIG. 2D. In an embodiment, the angle measurement 204 formed between the fixed arm 206 and moving arm 207 for the left thoracolumbar rotation may be stored alongside other recorded angle measurements corresponding to other ROM tests, for the generation of a comprehensive health report for the patient. It should be understood that both the right and left thoracolumbar rotation stretching procedures may simply be referred to as “thoracolumbar rotation stretching procedures”, as the process used for acquisition the left and right side ROM measurements is simply mirrored.

As a result of the described key-point identification and tracking method utilized to measure the ROM angles for a stretching procedure, wearable sensors and other peripheral technologies are not required. By simply utilizing a device with a camera and an internet connection to capture all of the necessary raw data to determine corresponding ROM values, the disclosed range of motion measurement process may be done in nearly any environment. As disclosed hereinabove, the herein disclosed process may utilize two-dimensional pose estimation to gather and generate relevant results from a captured recording, such as accurate ROM data for a patient 200, in an unbiased manner. Furthermore, this particular process can be executed virtually through telehealth enabled devices and its corresponding ROM measurements can be captured and recorded remotely with a high degree of accuracy through verifiable images/frames. As disclosed herein, the disclosed process utilizes the MMH system to measure a series of ranges, key-points, and lengths, then translates them into digitized data points, and measures the movement of these data points using a complex methodology, thus providing the desired ROM angles for a particular stretching procedure using only captured images/frames from the video recording. By utilizing the disclosed ROM measurement process, a patient may be provided with a fast and easy mechanism for determining their range of motion of a particular stretching procedure remotely and without having a clinician on site.

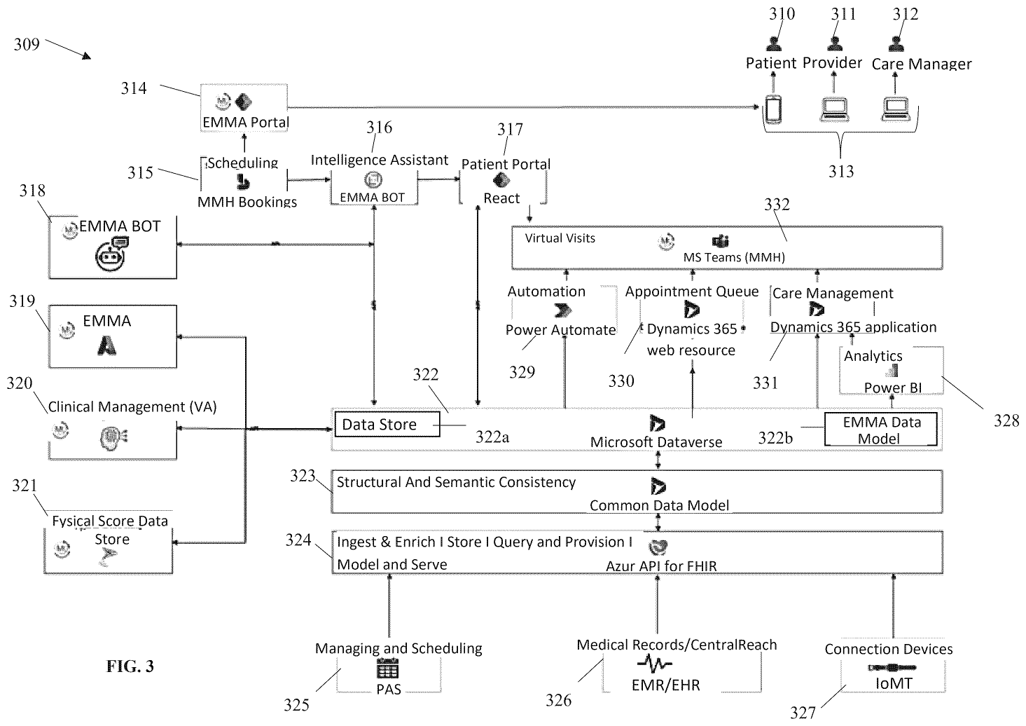

FIG. 3 illustrates the design diagram for the MMH system 309, according to an aspect. It should be understood that each arrow depicted interconnecting the various systems, databases, users, etc. of the MMH system 309 indicates that each of the disclosed elements are in data communication with each other. A singular arrow may be used to represent data being pulled from an element at the tail of the singular arrow to an element at the arrowhead of the singular arrow, whereas double arrows indicate the two elements may push and pull data to and from each other. It should be understood that while the disclosed design diagram of FIG. 3 may include specific programs, modules and systems, such as the Microsoft Dataverse 322, variations of each, such as other storage platforms, may be implemented in their place, as discussed hereinbelow.

Patients 310, providers 311 and care managers 312 may access the MMH System 309 by accessing an EMMA portal (“main portal”) 314 through the utilization of a suitable web-enabled device 313, such as a smartphone, tablet or computer. It should be understood that the EMMA portal 314 may be the same for the patient 310, provider 311 and the care manager 312, but said EMMA portal 314 may provide only the information relevant to or allowed for said user, based on their role. Said EMMA portal 314 may pull data from the scheduling module 315 responsible for indicating relevant MMH bookings and appointments to the user.

Said scheduling module 315 may be in data communication with the intelligence assistant 316 provided by the EMMA bot storage database (“Bot database”) 318. This intelligence assistant 316 may be responsible for asking patients questions and collecting results, including both initial and follow-up questionnaires, and prompting patients with relevant questions and information. The scheduling module 315 may make use of an existing scheduling software such as Microsoft Teams to provide a scheduling database. The intelligence assistant 316 may push data to a patient portal 317, which may act as a patient interface with the EMMA portal for patients to access relevant information pertaining to their health and other medical information, perform and review assessments, etc. In addition to its intercommunication with the scheduling module 315 and the patient portal 317, the intelligence assistant 316 may be in data communication with both the EMMA bot storage database 318 and the Microsoft Dataverse 322 in order to allow the AI assistant EMMA bot 316 to provide a user, such as a patient 310, with necessary information.

The Microsoft Dataverse 322 may act as a data hub, behaving as a central system component through which relevant information may be pushed and pulled to both collect and store on databases and access for usage from said databases. The Microsoft Dataverse 322 may be in direct data communication with the internal database, including the EMMA bot storage database 318, the EMMA AI/ML storage database 319, clinical management storage database 320, and the Injury Risk Index Score data store database 321, thus allowing information to be pushed and pulled to and from the internal database quickly and easily. As can be seen in FIG. 3, the Microsoft Dataverse 322 may also be in data communication with a common data model 323 and by extension the Azure API for FHR 324 in order to facilitate data transfer from external databases. It should be understood that the Azure API 324 may be provided as a potential embodiment of the API configured to access external databases, and that any API configured to perform this operation may be used, as will be described in greater detail hereinbelow.

These external databases may include a managing and scheduling database 325, a medical records/CentralReach database 326 and a IoMT (“internet of medical things”) database 327. Information acquired from these external databases may be helpful to supplement the information acquired through the conducted assessments and questionnaires, as having a knowledge of a patient's medical history may help provide a more precise indication of which follow up tests and assessments should be performed. A storage platform, provided as the Microsoft Dataverse 322 in the herein disclosed embodiment, may include two subcomponents: a datastore 322a in which pertinent information may be stored when being transferred between the various interconnected databases and interfaces and an EMMA data model 322b.

In contrast to the external database, each of the sub-databases of the internal database may be responsible for holding information directly relevant to the inner workings of the MMH system 309 and its generated reports. In addition, confidential information pertaining to system operations may be stored on these internal databases to suitably provide additional safeguards from it being accessed externally. The EMMA AI/ML (machine learning) database 319 may be responsible for storing information pertinent to the machine learning algorithms, and the overall function of the EMMA AI. The Clinical management (Virtual assistant) database 320 may contain data and a decision tree necessary for the operation of the virtual assistant EMMA bot 316 and reporting, and may contain lists of possible questions, prompts and exercise instructions. The Injury Risk Index Score Data Store database 321 may include processed information, such as patient Injury Risk Index Scores, and the information immediately relevant to its generation and other transactional data. It should be understood that the information required to conduct assessments, store and process the assessments and utilize the assessments to provide Injury Risk Index Score may be divided between the EMMA AI/ML (machine learning) database 319, the Clinical management (Virtual assistant) database 320 and the Injury Risk Index Score Data Store database 321.

The Microsoft Dataverse 322 may push information to a variety of other automation and processing based utilities, including an automation program capable of building and enabling automated processing, such as Power Automate 329, a queuing and organization application capable of providing the appointment queuing and care management practices, such as Dynamics 365 Applications 331 alongside Dynamics 365 Web Resource Systems 330, and an analytics generation program that provides the necessary analytics generation and processing capabilities utilized in the generation of medical reports, processed medical data, such as Power BI 328. Again, while the disclosed embodiment may describe specific programs, applications and modules that may be utilized to achieve the necessary functionalities, it should be understood that any program/application/module suited to perform the same tasks may be implemented in their stead. Power BI 328 may push its processed analytics into the care management system to allow their subsequent presentation. Each of these modules may then push their generated information to MS teams 332 to allow for its utilization during virtual visits and other relevant applications. It should be noted that MS Teams 332 may also pull information from the patient portal as needed for the virtual visit.

It should be understood that while the disclosed embodiment of FIG. 3 may identify capable Microsoft programs, applications and tools that may be used to enable system functionality, equivalent alternatives may be employed as long as the same functionalities are provided. The Microsoft Dataverse 322 and common data model 323 may be replaced by any situatable storage platform to provide the necessary functionality, while the Azure API of FHIR may also be replaced by any suitable API that allows for communication between the storage database and the external databases.

Similarly, Power Automate 329 may be replaced by any suitable automation program capable of building and enabling automated processing. In an embodiment, Dynamics 356 web resources 330 and the Dynamics 365 application 331 may be replaced with any suitable system capable of providing the appointment queuing and care management practices utilized by the MMH system. In an embodiment, Power BI 328 may suitably be replaced by other analytics generation processes that provide the necessary analytics generation and processing capabilities utilized in the generation of medical reports, processed medical data, etc. Teams 332 may also be replaced by any suitable web conferencing infrastructure that would facilitate virtual visits, such as telemedicine communications, between a patients and care providers, In short, the specific embodiments provided in FIG. 3 that are specific to a certain program or company (e.g., Microsoft) should be understood to indicate the potential for using alternative embodiments of each program, system or module that are provided as a component of the MMH system.

The overall structure of the disclosed MMH system 309 may be summarized succinctly by categorizing certain related elements into subcategories of said MMH system. The EMMA bot storage database 318, the EMMA AI/ML storage database 319, the clinical management storage database 320 and the Injury Risk Index Score Data Store database 321 may be grouped together and classified as an internal database, wherein said internal database is configured to securely store patient health information and data pertinent to the operation of the AI bot, Injury Risk Index Score calculations and report generation. The scheduling database 325, a medical records/CentralReach database 326, and the IoMT database 327 may be grouped together and classified as an external database, wherein said external database is configured to securely provide access to pertinent medical information not generated by the MMH system, but that is needed to determine a more complete medical history for patients.

In order to simplify the MMH system components utilized for interconnecting the various subsystems as a hub, the Microsoft Dataverse 322, Common data model 323, and 324 Azure API for FHIR may together be classified as central database, wherein said central database is responsible for facilitating data communication between the internal and external database, as well as the AI bot and a fourth subcategory of components referred to as a processing and communication module. Again, it should be understood that all modules depicted in the figures and described herein by their particular commercial name (e.g., Microsoft Dataverse 322, etc.) are provided solely for exemplification purposes. These modules may be substituted with functionally equivalent modules (e.g., Storage platforms, etc.) from other providers (e.g., Amazon).

The aforementioned processing and communication module may comprise the Power BI 328, Power automate 329, Dynamic 365 web resource and Dynamic 365 applications, or as described above, their utilized functional equivalents. This aforementioned processing and communication module may be in data communication with the patient portal as well as the central database. It should be understood that the prior described data communication between the user device 313, EMMA portal 314, scheduling module 315, AI bot 316, and the patient portal 317 may be maintained with the described MMH system subcategories, such that the EMMA portal is in data communication with the internal database and the central database, and the patient portal is in data communication with the processing and communication module.

Overall, the disclosed MMH system 309 makes use of both internal and external databases containing both preexisting and newly generated information in order to create reports and track patient progress through a medical care episode. The disclosed MMH system 309 may also take full advantage of the available Microsoft systems including Teams 332, Power Automate 329, the Microsoft Dataverse 322, Azure API for FHR 324, etc., in order to realize a semi-autonomous health care platform configured to supplement the existing physician facilitated care system that is currently utilized.

One benefit of using the segregated/partitioned data structure displayed within FIG. 3, wherein a plurality of separate databases is used, each of which houses a different type of information, is that information may be easily accessed by querying a smaller database having the needed information, rather than the larger database having all of the stored information. One result of this is that the data may be accessed and utilized more rapidly as a result of the reducing the processing power required to access it, thus expediting report generation. By reducing the processing power required to access and acquire the necessary data, additional system capabilities may be enabled to run simultaneously with reduced overall process load.

Depending on the signal strength available to a user in an area, the MMH system 309 may be configured to adapt to the user's environment. In an embodiment, the MMH system 309 may be configured to allow the user device, such as a mobile device, to handle a portion of the information processing prior to data transmission. This may significantly expedite data processing in instances in which the user's device has a poor internet connection by preprocessing the collected raw data from assessments, tests, etc. on the user's device, allowing only the smaller file size of the processed data to be forwarded over the internet to expedite data transmission times.

It should be understood that each internal database, including the EMMA bot storage database 318, the EMMA AI/ML storage database 319, clinical management storage database 320, and the Injury Risk Index Score data store database (“risk value database”) 321, may both push and pull data from the Microsoft Dataverse 322 in order to facilitate the required data transfer to enable system functionality as described herein. As disclosed hereinabove, by segregating/partitioning these databases, their information may be accessed more rapidly, expediting report generation while using less processing power than if all of the information were stored on a singular storage database.

One of the key benefits of the disclosed MMH system 309 is the automation that it enables within the preexisting health care infrastructure. Medical assessments and reports may be generated automatically without user or provider intervention. Procedures such as surgeries and other treatments may still require human intervention, but the automation enabled by the MMH system's ability to collect, process and utilize pertinent medical information allows it to significantly expedite diagnosis, interventions, and treatment in many instances.

As is understood, the MMH system 309 may be configured to interface with patients in order to collect the necessary information to determine their range of motion for a particular stretching procedure, such as the thoracolumbar rotation stretching procedures described in FIG. 1A-2D. Furthermore, in an embodiment, the MMH system 309 may also be configured to use an AI bot, such as the described EMMA bot 316, to instruct users through the stretching procedure and ensure the stretching procedure has been performed properly. Alternatively, as described hereinabove, a provider logged into the MMH system 309 may instead be providing instructions and confirming accuracy for the stretching procedures performed by the patient, in situations in which use of the EMMA bot for user instruction is not desired. In an embodiment, the MMH system itself is configured to record the necessary video of the patient performing the stretching procedure, extract the necessary frames from the video (such as at least the resting pose and the fully stretched pose), identify the key-points on the patient within the frames, trace the movement of said key-points, calculate the range of motion for the patient for that particular stretching procedure, record this resultant range of motion value in the corresponding database, and then report this range of motion to the appropriate party, such as the clinician/physician or the patient themselves.

As described hereinabove, each user of the MMH system 309 may have access to only the information relevant to them. For example, a patient's range of motion value for their right thoracolumbar rotation range of motion measurement test may be made accessible to the patient and their corresponding doctor/clinician/physician through the EMMA portal 314, whereas other users will not have access to the patient's information. This will allow medical information, such as the results for the patient's range of motion test, to only be accessed by authorized parties. It should be noted that the term “medical personnel” may be used to describe a doctor, clinician, physician, or other medical professional who may interface with the patient directly or may receive their test results/ROM values.

It should also be understood that the patient may interface with the MMH system 309 through a corresponding device, such as a computer, smartphone, tablet, or other device. Furthermore, in order to facilitate the recording of a video for a patient, in a preferred embodiment, the device will have a camera. In an alternative embodiment, a separate camera may be secured to the device in order to facilitate recording of stretching procedure videos for devices that do not include a camera. In an embodiment, the recorded range of motion values for a particular stretching procedure for a patient may be stored within a suitable database, such as the internal database, for later access by the patient or a corresponding physician.

It may be advantageous to set forth definitions of certain words and phrases used in this patent document. The term “couple” and its derivatives refer to any direct or indirect communication between two or more elements, whether or not those elements are in physical contact with one another. The term “or” is inclusive, meaning and/or. As used in this application, “and/or” means that the listed items are alternatives, but the alternatives also include any combination of the listed items.

The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. The terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases.

Throughout this description, the aspects, embodiments or examples shown should be considered as exemplars, rather than limitations on the apparatus or procedures disclosed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples.

What is claimed is:

1. A method of remotely measuring a patient's thoracolumbar rotation range of motion using a device having a camera, the device being in data communication with a computer system, the computer system having a database and an AI bot in data communication with the database, the method comprising:
instructing the patient through a thoracolumbar rotation stretching procedure by providing verbal instructions from the AI bot to the patient;
recording a video of the patient as they proceed through the stretching procedure, the video having at least a frame of the patient in a rest pose, a frame of the patient in a fully stretched pose and a frame of the patient in an intermediate pose between the rest pose and the fully stretched pose;
capturing the frames of the patient in the rest pose, the fully stretched pose and the intermediate pose from the video;
identifying key-points within the frames, the key-points comprising at least a corresponding acromion process of the patient;
tracing range of movement for the key-points in the frames;
calculating a range of motion angle for the stretching procedure by comparing key-point positions between the frames;
recording the range of motion angle in the database; and
reporting the range of motion angle to the patient or medical personnel.

2. The method of claim 1, wherein the patient performs the stretching procedure without the assistance of medical personnel.

3. The method of claim 1, further comprising observing the patient performing the stretching procedure, and requesting the stretching procedure be repeated if the patient performed the stretching procedure improperly.

4. The method of claim 1, wherein the stretching procedure is a right thoracolumbar rotation range of motion stretching procedure.

5. The method of claim 4, wherein the key-points comprise at least a right acromion process of the patient.

6. The method of claim 1, further comprising analyzing the frame of the patient in an intermediate pose for drift.

7. A method of remotely measuring a patient's range of motion using a device having a camera, the device being in data communication with a computer system, the computer system having a database and an AI bot in data communication with the database, the method comprising:
instructing the patient through a stretching procedure;
recording a video of the patient as they proceed through the stretching procedure, the video having at least a frame of the patient in a rest pose and a frame of the patient in a fully stretched pose;
capturing the frames of the patient in the rest pose and the fully stretched pose from the video;
identifying key-points within the frames;
tracing range of movement for the key-points in the frames;
calculating a range of motion angle for the stretching procedure by comparing key-point positions between the frames;
recording the range of motion angle in the database; and
reporting the range of motion angle to the patient or medical personnel.

8. The method of claim 7, wherein the patient performs the stretching procedure without the assistance of medical personnel.

9. The method of claim 7, wherein instructing the patient through the stretching procedure is performed by an AI bot communicating with the patient through the device.

10. The method of claim 9, further comprising observing the patient performing the stretching procedure, and requesting the stretching procedure be repeated if the patient performed the stretching procedure improperly.

11. The method of claim 7, further comprising observing the patient perform the stretching procedure to ensure that the stretching procedure is performed properly, wherein instructing the patient through a stretching procedure is performed by medical personnel in communication with the client through the device.

12. The method of claim 7, wherein the stretching procedure is a thoracolumbar rotation range of motion stretching procedure.

13. The method of claim 12, wherein the key-points comprise at least a left acromion process of the patient.

14. The method of claim 7, further comprising capturing a frame of the patient in an intermediate pose, wherein the video further comprises the frame of the patient in the intermediate pose, and analyzing the frame of the patient in an intermediate pose for drift.

15. A method of remotely measuring a patient's range of motion using a device having a camera, the device being in data communication with a computer system, the computer system having a database, the method comprising:
instructing the patient through a stretching procedure;
recording a video of the patient as they proceed through the stretching procedure, the video having at least a frame of the patient in a rest pose and a frame of the patient in a fully stretched pose;
capturing the frames of the patient in the rest pose and the fully stretched pose from the video;
identifying key-points within the frames;
tracing range of movement for the key-points in the frames;
calculating a range of motion angle for the stretching procedure by comparing key-point positions between the frames;
recording the range of motion angle in the database; and
reporting the range of motion angle to the patient or medical personnel.

16. The method of claim 15, wherein the patient performs the stretching procedure without the assistance of medical personnel.

17. The method of claim 15, the computer system further comprising an AI bot in data communication with the database, wherein instructing the patient through the stretching procedure is performed by an AI bot communicating with the patient through the device.

18. The method of claim 17, further comprising observing the patient performing the stretching procedure, and requesting the stretching procedure be repeated if the patient performed the stretching procedure improperly.

19. The method of claim 15, further comprising asking the patient intake questions.

20. The method of claim 15, further comprising capturing a frame of the patient in an intermediate pose, wherein the video further comprises the frame of the patient in the intermediate pose and analyzing the frame of the patient in an intermediate pose for drift.
